(19) United States
(12) Patent Application Publication
(10) Pub. No.: US 2006/0270960 A1
(43) Pub. Date: Nov. 30, 2006

(54) Treatment of blood with light

(76) Inventor: Nelson M. Karp, Virginia Beach, VA (US)

Correspondence Address: WILLIAMS MULLEN, 222 CENTRAL PARK AVENUE, SUITE 1700, VIRGINIA BEACH, VA, 23462-3035, US

(21) Appl. No.: 11/497,789

(22) Filed: Aug. 2, 2006

Related U.S. Application Data
(63) Continuation-in-part of application No. 10/462,344, filed on Jun. 16, 2003.
(60) Provisional application No. 60/388,798, filed on Jun. 14, 2002.

(51) Int. Cl. A61M 37/00

(52) U.S. Cl. 604/6.08

(57) ABSTRACT

A device for the exposure of blood to light produces a controlled, safe, and rapid exposure of blood to specific emissions, thereby inducing improved immune response. The device exposes the blood through a simplified macro blood flow path in which the internal or micro blood flow is in a spiral motion. The device includes a blood flow path, a pump, an exposure chamber, an ultraviolet light source, and a vacuum chamber.

### CROSS REFERENCE TO RELATED APPLICATION

The present application claims priority from U.S. patent application Ser. No. 10/462,344 filed Jun. 16, 2003, which claims benefit of U.S. Provisional Application Ser. No. 60/388,798, filed Jun. 14, 2002.

### BACKGROUND OF THE INVENTION

1. Field of the Invention

The present invention relates to an apparatus and method for the exposure of blood to light as a medical treatment. More specifically, the invention involves the controlled exposure of a stream of blood to ultraviolet (UV) light under conditions that effect a salubrious change in the blood.

2. Description of the Related Art

Light is well known as an effective medical treatment. In the nineteenth century, it was shown that light could inhibit bacteria growth and kill some microorganisms. In 1903, Niels R. Finsen won the Nobel Prize for Medicine by treating certain skin conditions (e.g., tuberculosis) with light. Before the advent of antibiotics, physicians began to use UV light to treat a variety of infections, many of which were ill defined at the time.

In the 1920's, 1930's, and 1940's, researchers began to develop devices for the exposure of blood to UV radiation, and reported positive results. For example, U.S. Pat. Nos. 1,683,877, 2,308,516, and 2,309,124 to L. A. Edblom and E. K. Knott are considered by some to be the key, early developments in the field, which was known as the Knott HEMOIRRADIATOR®. Although the mechanism of this device and the treatment were little understood at the time, the conclusion was that UV blood irradiation therapy enhanced the body's immune response. These references disclosed an extra-corporeal system in which whole blood was drawn, mixed with an anti-coagulant, pumped through a chamber where it was exposed to UV light between 1800 and 4000 angstroms—with a concentration or peak at 2540 angstroms—and then returned to the body. Although the first invention disclosed blood flow through two needles, Knott found a single needle arrangement to be speedier for his closed loop system. The exposure chamber included a transparent window through which the light source would shine onto the flowing blood. The chamber was designed to agitate the blood as it flowed by this flat window, so that more cells and bacteria would be exposed to the UV light. The '516 patent refined the device and advised users that exposure of more than 5 seconds could be detrimental.

Historically, the introduction of antibiotics and vaccines reduced the interest in the use of light for medical treatment. Nevertheless, development continued throughout the twentieth century. Most of the later developments seemed to be characterized by inventions that involved: (a) the separation and exposure of a portion or component of the blood (e.g., U.S. Pat. No. 4,613,322 to Edelson), (b) the addition of a compound or photo-active agent to the blood (e.g., U.S. Pat. No. 4,737,140 to Lee), or (c) both (e.g., U.S. Pat. Nos. 4,321,919, 4,398,906, 4,464,166, 4,612,007, 4,613,322, 4,683,889, 4,684,521 all to Edelson). A few references addressed improvements in the design of the exposure chamber and the blood transport system. As in the Knott design, all these developments retained the need for anti-coagulant treatment, and focused on large or macro scale improvements in exposure.

In U.S. Pat. No. 5,150,705, Stinson disclosed a cylindrical exposure chamber comprising a central UV light source, effective for treating transplant cells, located within a UV transparent cylinder, and UV transparent tubing for carrying a cellular suspension. The tubing is wrapped helically about the cylinder to promote consistent exposure of the tubing to the UV source. Such a macro scale arrangement was intended to maximize the tube's efficiency in capturing UV emissions from a cylindrical source. However, depending on the overall fluid characteristics, the micro scale blood flow relative to the tube could be stratified by density, leading to uneven exposure of the blood.

Another example of an evolutionary configuration of an exposure chamber is shown in U.S. Pat. No. 6,312,593, to Petrie. The Petrie device discloses a chamber that features a series of baffle plates and transverse protuberances to produce a Bernoulli distortion in the blood flow, creating desirable agitation. In fact, the Knott design contemplated agitation of the blood; this invention used the energy of the fluid flow reacting to a transverse disturbance to cause a pressure gradient, thereby forcing blood at a lower depth to move upward. As the blood moved up, it had a greater chance of being exposed to the flat transparent window that Knott introduced. The transverse aspect of the disturbance required sufficient longitudinal flow to generate enough disturbance for the desired exposure.

Accordingly it is an object of the present invention to provide a method for treating blood which provides improved exposure of blood to UV light.

Another object of the present invention is to provide a method for treating blood which increases the likelihood that each blood cell is exposed to the proper amount of UV light.

Another object of the present invention is to provide a method for treating blood which reduces the amount time blood must be exposed to UV light.

Another object of the present invention is to provide a method for treating blood which reduces the likelihood of cellular separation and hemolysis.

Yet another object of the present invention is to provide a method for treating blood which treats blood rapidly.

Yet another object of the present invention is to provide a method for treating blood which does not result in coagulation of the blood.

Finally, it is an object of the present invention to accomplish the foregoing objectives in a simple and cost effective manner.

### SUMMARY OF THE INVENTION

The present invention addresses the foregoing problems, as well as other problems, by providing a chamber for exposing a stream of blood flowing through the chamber to ultraviolet light to destroy microorganisms and to stimulate the immune system. The chamber includes an ultraviolet-light-transparent blood flow path having an inlet port and an outlet port. The blood is induced to flow in a spiral within the blood flow path located within the chamber and is exposed to an ultraviolet light source. The blood flow path can be formed from any ultraviolet-light-transparent material such as polystyrene, polypropylene or quartz. If desired, the blood flow path can be formed from a plurality of materials, such that different portions absorb a different wavelength of UV light or, alternatively, one or more filters can be used to regulate the exposure of the blood flow path to ultraviolet rays. The spiral movement in the blood is caused by, for example, threaded walls on the internal surface of the blood flow path or by a static mixer. Preferably, a vacuum is formed between the ultraviolet light source and the blood flow path. Any gaps between the ultraviolet light source and the blood flow path can be filled with an optically transparent material such as quartz. The ultraviolet light source can be one or more light bulbs, preferably emitting light within at least the ultraviolet A range. If appropriate, the ultraviolet light can be pulsed such that the blood is exposed to the ultraviolet light on a discontinuous basis.

### DESCRIPTION OF THE DRAWINGS

FIG. 1 is a view of the flow path used in the preferred embodiment of the present invention;

FIG. 2 is a diagram showing the top view of an embodiment;

FIG. 3 is a method of using the embodiment of the present invention;

FIG. 4 is a diagram showing a particularly preferred embodiment of the present invention;

FIGS. 5 and 6 show examples of static mixers;

FIG. 7 is an example of spiral blades;

FIGS. 8 and 9 are end views of examples of blood flow paths having internal threads, while 10 shows an exterior of a blood flow path having internal threads; and

FIG. 11 shows a top view of an embodiment of the present invention.

### DETAILED DESCRIPTION OF THE PREFERRED EMBODIMENT

The following detailed description is of the best presently contemplated modes of carrying out the invention. This description is not to be taken in a limiting sense, but is made merely for the purpose of illustrating general principles of embodiments of the invention.

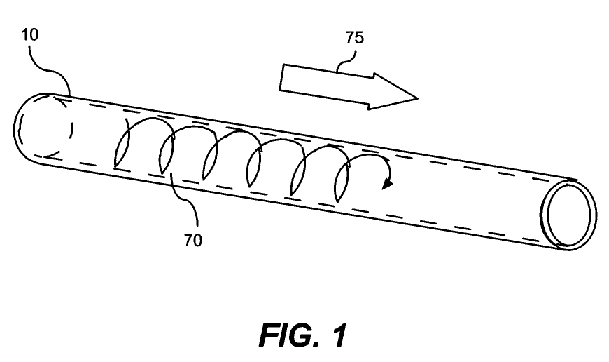

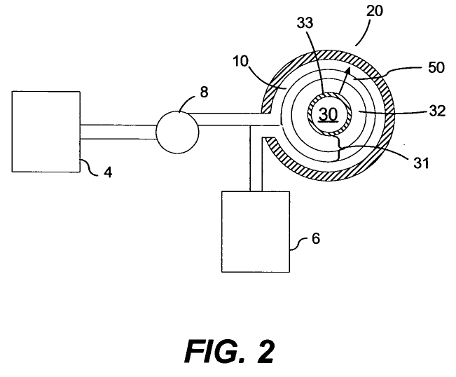

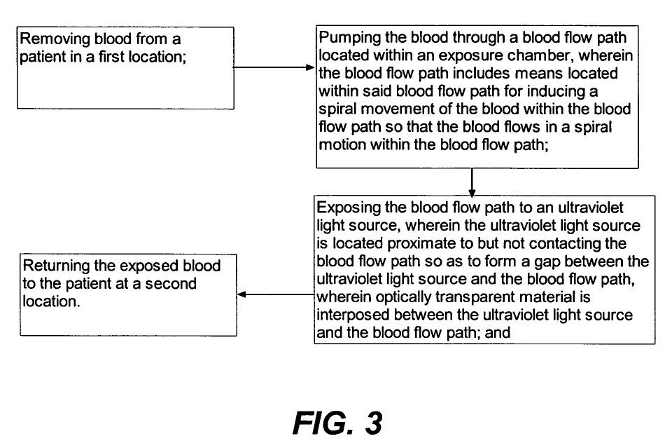

A device for the exposure of blood to light can be built that produces a controlled, safe, and rapid exposure of blood to specific emissions, thereby inducing improved immune response. The present invention achieves this through a simplified blood flow path. FIG. 1, on a micro scale, includes arrow 70 demonstrating the spiral flow of blood within blood flow path 10, along the longitudinal direction shown by arrow 75. On a macro scale, FIG. 2 shows the invention, which includes a blood flow path 10, an exposure chamber 20, a light source 30, and optionally wherein the exposure chamber 20 is vacuum tight. Also shown is a collection 4 of blood withdrawn from a patient, a deposit 6 of blood for return to a patient, and a pump 8, for circulation. The preferred embodiment contemplates an UV light source 30 producing light 50. Preferably, the invention is of a flow-through design.

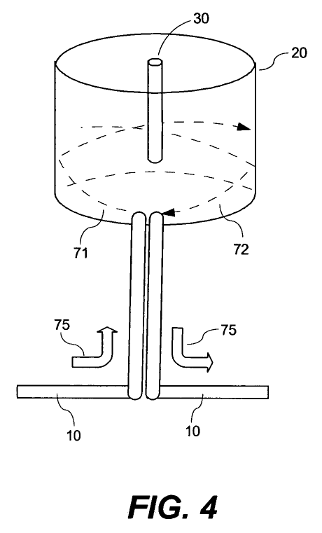

The exposure chamber design enhances control over the exposure of the blood to ultraviolet light. The exposure chamber includes a blood flow path 10, such as a modified tube, or channel crafted from a UV transparent material, such as transparent polystyrene or polypropylene. These materials permit blood flow in a variety of macro configurations or arrangements during the period of exposure to UV light, so long as each red blood cell is exposed to the emissions for the desired time. In the event that an otherwise desirable transparent material is found to absorb an important wavelength of UV light, then the flow path could be constructed of segments of varying light absorption characteristics (i.e., different transparent materials). Alternatively, filters may be employed to expose the blood to selected wavelengths. A particularly preferred design for the macro configuration within the exposure chamber 20 is a double helix type geometry as shown in FIG. 4. Those of skill in the art may distinguish between the macro configuration (or circuit) of the blood flow path 10 and the micro or internal flow characteristics of blood within blood flow path 10. Arrow 71 shows blood flow path 10 in exposure chamber 20 on a macro scale, helically ascending and arrow 72 shows blood flow path 10 helically descending. In this embodiment, the UV light source 30 is positioned in the center of the exposure chamber 20. This design results in particularly consistent and uniform exposure of the blood to the UV light and minimization of hemolysis.

Consistent exposure of all or most of the subject cells is safer and more efficient than variable, partial exposure. A spiral or helical path of tubing on a macro scale has been tried in order to enable a tube to embrace a generally cylindrical UV source and to capture efficiently its emissions. That design concentrated on efficient use or capture of the UV emissions by overall configuration of the tubing. In contrast, as shown in FIG. 1, a spiral flow path on a micro scale can produce the internal flow characteristics and external chamber geometry useful to enhance micro scale control regardless of the macro scale design. In the present invention, the blood flow is rendered helical or spiral within (i.e., internally) and relative to the blood flow path 10, as shown in FIG. 1, and then the blood flow path 10 can be configured about an ultraviolet light source 30 (not shown) for the desired exposure.

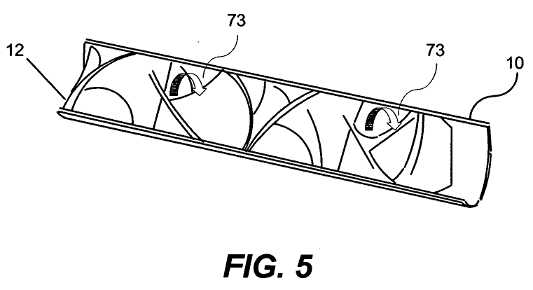

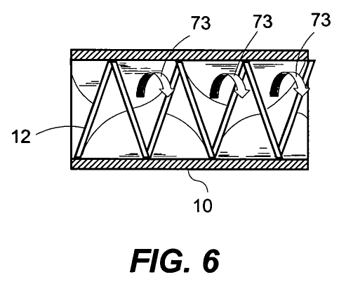

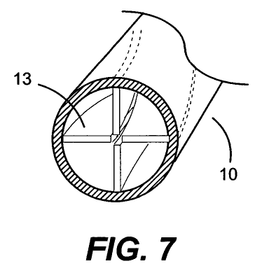

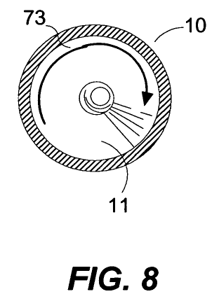

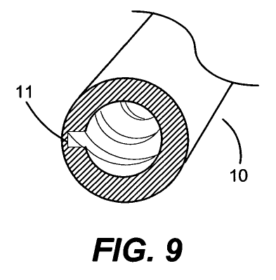

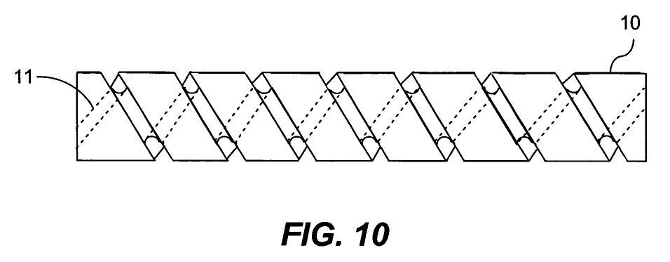

The desired internal spiral flow of blood within blood flow path 10 may be achieved in a variety of ways. This is not the same thing as a macro scale orientation or potentially helical twisting of a flow path 10. Instead, this is a channel or tube featuring means to induce an internal or micro scale spiral flow; the blood flow path 10 may feature means for inducing a spiral flow of the blood such as internally threaded walls, a spiral blade, or an internal Kenics® static mixer. Kenics® static mixers are a class of mixers that use static structural elements 12, such as vanes situated within a flow channel for alternating or changing fluid flow. An example of a static mixer is shown in U.S. Pat. No. 3,286,992. Some versions of these mixers, as shown in FIGS. 5 and 6, have helical vanes for their static structural elements 12 that are capable of inducing a spiral or helical flow or movement of liquid within a tube. As blood flow within the blood flow path 10 encounters a helical static structural element 12, the direction of the flow is changed from merely longitudinal to an internal spiral, as shown with arrow 73, along the longitudinal. Thus, the helical static structural elements 12, spiral blades 13, or the internal threads 11 need be of sufficient size and configuration so as to redirect the longitudinal flow to induce a spiral flow of the blood along the longitudinal. A combination of a plurality of such static structural elements 12 of a mixer is shown in examples of FIGS. 5 and 6. One or more spiral blades 13 are shown in a four blade example in FIG. 7. Examples of internal threads 11 are shown in examples FIGS. 8-10. All these examples may thus be configured to induce a spiral flow of blood internally within the blood flow path 10 preferably while in the exposure chamber 20 (not shown), regardless of the external or macro configuration. The internal production of a spiral flow of blood within a blood flow path 10 is adaptable to 360-degree exposure, either directly or through reflection. The frequency of the spiral rotation may be segmented or metered for the desired exposure, at a given a chamber size, source, and flow rate. Although different applications may drive a final configuration, such a spiral flow enables greater control of the exposure to of the sample volume. The blood cells are spiraled or mixed internally during their longitudinal travel along the flow path, and are therefore more likely to be exposed to the UV emissions during their transit of a given length of flow path. A predictable level of exposure per longitudinal unit means that the overall time of exposure may be reduced. Thus, the macro or external configuration of the blood flow path 10 may be straight, double helical, or other pattern so long as the macro or external configuration does not interfere with the micro or internal spiral flow. Those skilled in the art of blood flow dynamics will readily see the alternative configurations available.

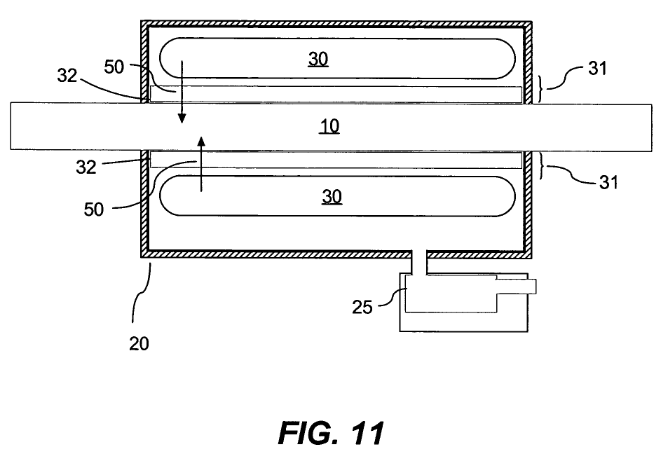

Preferably, for the macro scale design, as illustrated in the simple example of FIG. 11, the distance or gap 31 defined between the ultraviolet light source 30 and the blood flow path 10 within the chamber is minimized. Any gap 31 (preferably less than 3 cm) between the blood and the ultraviolet light source is filled with an optically transparent material 32, such as quartz. Further, preferably any larger gaps 31 or voids in the exposure chamber 20 between the blood flow path 10 and the ultraviolet light source 30 not filled with optically transparent material 32 should preferably be maintained at a vacuum to avoid ionization of the air and variation in the characteristics of the radiation. This may be achieved in a variety of conventional ways. For example, the exposure chamber 20 may be sealed or air tight. A vacuum pump 25 or similar device may be applied to evacuate the exposure chamber 20.

The light source 30 should provide the desired spectral emissions, with exposure appropriate to the flow and configuration of the chamber. The UV light source 30 can be a single UV light bulb or a plurality of UV light bulbs. Preferably, a light source 30 emitting UVA light is used. If a light bulb, or combination of light bulbs, which emits UVB or UVC light in addition to UVA light is used, one or more filters 33 can be used so that the blood is exposed to the desired UVA light, as shown in FIG. 2. Ideally, the blood is exposed to UV light having peak wavelengths of 365 nm and 254 nm. Designs based on the Knott device would provide a range of UV wavelengths from 2000 to 4000 angstroms, with an intensity ranging from 40 to 1,538 W/cm². Other designs, such as that in '566 to Schleicher, contemplate a wavelength output of 2,000 to 12,000 angstroms. The UV light source 30 may be pulsed or shuttered at a desired frequency; in this case, a pulse of approximately two to three hertz would be standard. As noted above, micro scale induction of spiral flow enables a wide variety of macro scale arrangements, so the blood flow path 10 can be structured to accommodate different types of light sources. To ensure that the blood is being exposed to the appropriate UV light, a spectrophotometer can be used to monitor and regulate, when appropriate, the UV emissions.

The design of the exposure chamber, the pulse frequency and intensity, and the volumetric flow rate are preferably considered together for optimal control. Many whole blood designs seek an individual red cell exposure period of about ¼ to ½ seconds, while also avoiding cellular separation and hemolysis. The volume of the exposure chamber will depend on the pulse frequency, desired exposure time, and volumetric flow rate. A sample of approximately 250 milliliters of blood (or 1.5 milliliters per kilogram of body weight) may be treated at a time. In a design with a volumetric blood flow rate of approximately 1 milliliter per second, the device could process the entire sample through the exposure chamber in a period of four minutes and ten seconds.

In a particularly preferred embodiment, the exposure chamber and blood flow path can be made as disposable items. This embodiment has the added benefits of eliminating the need to sterilize these items and the concern of transmission of infectious diseases.

An additional feature of the micro scale approach is reduced time of treatment. In the present invention, the spiral flow within the exposure chamber promotes controlled, thorough, and rapid exposure. Prior designs that operated more slowly face the problem of coagulation of the blood. Typical approaches to compensate for this problem are the addition of heparin, or the inclusion of other anti-coagulant measures. The rapidity and control of the present invention offers an alternative. Preferably, the blood should be drawn from one point and returned to the patient at a different point, as shown in FIG. 2. For example, the blood could be withdrawn from the antecubital fossa or other convenient venous access of the upper extremity of a left arm and, after treatment, returned to a symmetric location on the right arm. This quick, flow through design, combined with rapid exposure to the light source, reduces the chance of coagulation of the blood, so that an anti-coagulant step is not required. In fact, in its whole blood treatment embodiment, the present invention contemplates no need for any other additives, such as photopheretic compounds or other active agents.

In an alternate embodiment of the present invention, the blood can be separated into four portions once it is removed from the patient: erythrocytes, leukocytes, platelets and blood plasma. In this embodiment, each portion would be separately exposed to ultraviolet light for treatment and then reintroduced to the patient separately or collectively.

This contemplated arrangement for the exposure of blood to light may be achieved in a variety of configurations. While there has been described what are believed to be the preferred embodiment of the present invention, those skilled in the art will recognize that other and further changes and modifications may be made thereto without departing from the spirit of the invention, and it is intended to claim all such changes and modifications as fall within the true scope of the invention.

What is claimed is:

1. A device for exposing a stream of blood flowing through the device to ultraviolet light to destroy microorganisms and to stimulate the immune system, said device comprising:
a chamber;
an ultraviolet-light-transparent blood flow path within the chamber having an inlet port and an outlet port, suitable for containing a stream of blood;
a means located within said blood flow path for inducing a spiral movement of the blood within the blood flow path as said blood flows from the inlet port to the outlet port along the blood flow path; and
an ultraviolet light source disposed within the chamber proximate to but not contacting the blood flow path so as to define a gap between the ultraviolet light source and the blood flow path, and wherein the ultraviolet light source is adapted to expose the blood flow path with ultraviolet rays.

2. The device described in claim 1 wherein the blood flow path is formed from a material selected from the group consisting of polystyrene, polypropylene and quartz.

3. The device described in claim 1 wherein the blood flow path is formed from a plurality of materials, each material absorbing a different wavelength of UV light.

4. The device described in claim 1 further comprising at least one filter for regulating the exposure of the blood flow path to ultraviolet rays.

5. The device described in claim 1 wherein the spiral movement of the blood within the blood flow path is caused by threaded walls on the internal surface of the blood flow path.

6. The device described in claim 1 wherein the spiral movement of the blood within the blood flow path is caused by a static mixer.

7. The device described in claim 1 wherein the spiral movement of the blood within the blood flow path is caused by at least one spiral blade.

8. The device described in claim 1 wherein the chamber is evacuated by a vacuum source in fluid communication with the chamber, configured so as to be capable of evacuating air from the chamber to minimize ionization of the air.

9. The device described in claim 1 wherein optically transparent material is positioned within the gap interposed between the ultraviolet light source and the blood flow path.

10. The device described in claim 1 wherein quartz is positioned within the gap interposed between the ultraviolet light source and the blood flow path.

11. The device described in claim 1 wherein the ultraviolet light source consists of an ultraviolet light bulb emitting light in the ultraviolet A range.

12. The device described in claim 1 wherein the ultraviolet light source consists of a plurality of ultraviolet light bulbs.

13. The device described in claim 1 wherein the ultraviolet light source is pulsed such that the blood is exposed to the ultraviolet light on a discontinuous basis.

14. The device described in claim 1 wherein the blood flow path is configured in a helix about the ultraviolet light source.

15. A method for treating blood with ultraviolet light, comprising: removing blood from a patient at a first location; pumping the blood through a blood flow path located within an exposure chamber wherein the blood flow path includes means located within said blood flow path for inducing a spiral movement of the blood within the blood flow path so that the blood flows in a spiral motion within the blood flow path; exposing the blood flow path to an ultraviolet light source, wherein the ultraviolet light source is located within the exposure chamber proximate to but not contacting the blood flow path so as to define a gap between the ultraviolet light source and the blood flow path; and returning the exposed blood to the patient at a second location.

16. The method for treating blood described in claim 15 wherein the blood flow path is formed from an ultraviolet light transparent material.

17. The method for treating blood described in claim 15 wherein the spiral motion of the blood flow is induced by threaded walls on the internal surface of the blood flow path.

18. The method for treating blood described in claim 15 wherein the spiral motion of the blood flow is induced by a static mixer.

19. The method for treating blood described in claim 15 wherein the spiral motion of the blood flow is induced by at least one spiral blade.

20. The method for treating blood described in claim 15 wherein the chamber is evacuated by use of a vacuum source in fluid communication with the exposure chamber and in such configuration as to be capable of evacuating air from the exposure chamber, so as to minimize the ionization of the air.

21. The method for treating blood described in claim 15 wherein optically transparent material is positioned within the gap interposed between the ultraviolet light source and the blood flow path.
